(19) United States
(12) Patent Application Publication
(10) Pub. No.: US 2010/0287627 A1
(43) Pub. Date: Nov. 11, 2010

(54) Transgenic Zebrafish Models for Neurodegenerative Diseases

(76) Inventor: Amy L. Rubinstein, Smyrna, GA (US)

Correspondence Address: PATENT CORRESPONDENCE;ARNALL GOLDEN GREGORY LLP, 171 17TH STREET NW, SUITE 2100, ATLANTA, GA, 30363, US

(21) Appl. No.: 12/646,083

(22) Filed: Dec. 23, 2009

Related U.S. Application Data
(63) Continuation of application No. 12/274,271, filed on Nov. 19, 2008, now abandoned.
Application No. 12/274,271 is a continuation of application No. 10/116,723, filed on Apr. 4, 2002, now abandoned.
(60) Provisional application No. 60/281,347, filed on Apr. 4, 2001.

(51) Int. Cl. A61K 49/00; A61P 25/28

(52) U.S. Cl. 800/3

(57) ABSTRACT

The present invention relates to zebrafish models for neurodegenerative disorders that allow screening of compounds for their ability to protect and/or regenerate neurons in vivo in a whole vertebrate organism. The present invention also provides methods of identifying gene targets for neuroprotective compounds, compounds that regenerate neurons and compounds that promote neurogenesis.

This application claims priority to U.S. Provisional Application No. 60/281,347 filed on Apr. 4, 2001, which is hereby incorporated by this reference in its entirety.

### FIELD OF THE INVENTION

The present invention relates to zebrafish models for neurodegenerative disorders that allow screening of compounds for their ability to protect and/or regenerate neurons in vivo in a whole vertebrate organism. The present invention also provides methods of identifying gene targets for neuroprotective compounds, compounds that regenerate neurons and compounds that promote neurogenesis.

### BACKGROUND

As the American population ages, an increase in degenerative disorders of the nervous system, including Alzheimer's Disease, Parkinson's Disease, and amyotrophic lateral sclerosis (ALS, or Lou Gehrig's disease), is expected. These diseases affect greater than 4 million people in the United States, with a similar prevalence worldwide. Few treatment options exist for these debilitating diseases and those treatments that are available are of limited efficacy. A need exists for novel approaches to compound screening to identify new treatments for these diseases. Parkinson's disease is a progressive neurodegenerative disorder affecting over one million people in the United States. Symptoms include uncontrolled tremors, stooped posture and gait disturbances. Morphologically, Parkinson's disease is characterized by a loss of the pigmented dopaminergic neurons located in the substantia nigra, resulting in depletion of the neurotransmitter dopamine. Other groups of neurons, such as the noradrenergic neurons of the locus coeruleus, can also be affected. Formation of Lewy inclusion bodies in the substantia nigra is another hallmark of Parkinson's disease (reviewed by Zhang, et al., 2000).

Although the cause of Parkinson's disease remains elusive, genetic and other studies of Parkinson's patients have provided some clues. For example, rare cases of familial Parkinson's disease have been linked to mutations in two different proteins, alpha-synuclein and parkin. Although the function of these proteins remains unknown, they may be starting points for understanding the pathology of the disease. Alpha-synuclein has been found in Lewy bodies and the homology of the parkin gene to ubiquitin suggests a link to the ubiquitination pathway. Other studies of Parkinson's patients and animal models have indicated a role for oxidative stress in neuronal cell death (Zhang, et al., 2000).

The most common current treatments for Parkinson's disease focus on replacement of dopamine, either through direct administration of its immediate precursor, levodopa, or by administration of dopamine receptor agonists or inhibitors of dopamine metabolic enzymes, such as L-deprenyl (Grünblatt, et al., 2000). Although replacement of dopamine affords symptomatic relief, it does not slow the progression of the disease and patients often become refractory to treatment. Levodopa can also cause severe side effects. Therefore, there is a need for new drugs to treat this and other neurodegenerative diseases.

Another example of a neurodegenerative disorder is amyotrophic lateral sclerosis (ALS). ALS, also known as Lou Gehrig's disease, is a disorder characterized by progressive weakness and paralysis, eventually leading to death in 1-5 years. It affects approximately 5 per 100,000 persons (reviewed by Wong, et al., 1998). The underlying condition that results in these symptoms is the selective degeneration and death of spinal cord motor neurons. Abnormal accumulation of neurofilament proteins is also associated with the disease. Although clinical trials have been conducted to investigate the efficacy of certain neurotrophic factors, an effective treatment for ALS has yet to be found (reviewed by Mitsumoto, et al., 1999). Only one drug (riluzole, marketed by Aventis under the trade name Rilutek) is currently approved for treatment of ALS, and its effect is only modest (Hurko and Walsh, 2000).

Genetic studies have provided some information about the cause of ALS. Approximately 10% of ALS cases are inherited; of these 20% are due to mutations in the superoxide dismutase (SOD1) gene (Cleveland, 1999). This finding suggests that oxidative damage may play a role in the disease. Of sporadic cases of ALS, about two thirds were found to have low levels of a glutamate transporter, required for removing the high levels of the neurotransmitter glutamate that accumulate after synaptic transmission (Cleveland, 1999). Accumulation of glutamate is known to be toxic to neurons (Rothstein, et al., 1993; Annis and Vaughn, 1998). High levels of glutamate have been shown recently to slow transport of neurofilaments, and thus glutamate toxicity may also be related to abnormal accumulation of neurofilaments (Ackerley, et al., 2000). Riluzole, the only approved drug to treat ALS, is believed to act by inhibiting the spontaneous release of glutamate (Louvel, et al., 1997).

Although ALS affects a relatively small portion of the population, it represents a useful model for neuronal drug discovery, see Hurko and Walsh (2000). First, it affects a well-defined subset of neurons that can be easily quantified in experimental treatment protocols. In addition, treatments for ALS can be potentially useful for the treatment of other neurological diseases, since the underlying causes may be similar. Riluzole, for example, is currently in clinical trials for Parkinson's disease and Huntington's disease.

Mice that are mutant for SOD1 form the basis for the most widely used animal model of this disease (Cleveland, 1999). Other naturally occurring mouse mutants with motor neuron dysfunction, such as the wobbler mouse, have also been used, as well as the artificial induction of neuronal injury by axotomy (Elliot, et al., 1999). Differentiated motor neurons in cell culture have been of only limited use (Silani, et al., 2000).

Rat spinal cord explants have been more useful as a method to study motor neurons in culture. For example, explants have been used to test potential drug candidates for ALS (Corse, et al., 1999). In this model, motor neurons in cultured explants are destroyed by incubating them with threohydroxyaspartate (THA, a glutamate transport inhibitor). N-methyl-D-aspartate (NMDA), a glutamate agonist can also have this effect (Annis and Vaughn, 1998). NMDA-induced damage of motor neurons in culture can be ameliorated by coculture with the glutamate antagonist D-AP5 (Annis and Vaughn, 1998). Corse, et al. (1999) tested several neurotrophic factors for their ability to protect motor neurons from THA-induced excitotoxicity. Neurotoxicity induced in this way is especially relevant since many researchers hypothesize that human ALS is a condition resulting from glutamate excitotoxicity (Brown, 1997; Shaw and Eggett, 2000). However, none of the assays or models currently available allow for rapid screening and analysis of neuronal function in a whole vertebrate organism.

This invention allows characterization of the function of proteins, small molecules, drugs, chemicals and other compounds in neurons of zebrafish (Danio rerio) embryos. The methods described herein encompass the creation and use of transgenic zebrafish strains that express a reporter protein in specific subsets of neurons. Therefore, the present invention provides a way to rapidly screen thousands of proteins, small molecules, drugs, chemicals and other compounds for function and toxicity in a whole vertebrate organism. Furthermore, the present invention provides methods for the identification of neuron-specific drug targets for neuroprotectants, compounds that promote regeneration and compounds that promote neurogenesis.

### SUMMARY OF THE INVENTION

The present invention provides a method of identifying a compound that protects neurons comprising: a) contacting a transgenic zebrafish expressing a reporter protein in neurons with a neurotoxin and a test compound; b) comparing the expression of the reporter protein in the neurons of zebrafish contacted with the neurotoxin and the test compound with the expression of the reporter protein in the neurons of a transgenic zebrafish that was contacted only with the neurotoxin; c) determining the effect of the test compound on the expression of the reporter protein in the neurons, such that if the expression of the reporter protein in the neurons of the zebrafish contacted with the neurotoxin and the test compound is greater than the expression of the reporter protein in the zebrafish that was contacted only with the neurotoxin, the compound protects neurons from the neurotoxin.

Further provided by the present invention is a method of identifying a compound that regenerates neurons comprising: a) contacting a neuronally damaged transgenic zebrafish expressing a reporter protein in neurons with a test compound; b) comparing the expression of the reporter protein in the neurons of the zebrafish contacted with the test compound with the expression of the reporter protein in the neurons of a neuronally damaged transgenic zebrafish that was not contacted with the test compound; c) determining the effect of the test compound on the expression of the reporter protein in the neurons, such that if the expression of the reporter protein in the neurons of the zebrafish contacted with the test compound is greater than the expression of the reporter protein in the zebrafish not contacted with the test compound, the compound regenerates neurons in the neuronally damaged zebrafish.

Also provided by the present invention is a method of identifying a compound that promotes neurogenesis comprising: a) contacting a neuronally damaged transgenic zebrafish expressing a reporter protein in neurons with a test compound; b) comparing the expression of the reporter protein in the neurons of zebrafish contacted with the test compound with the expression of the reporter protein in the neurons of a neuronally damaged transgenic zebrafish that was not contacted with the test compound; c) determining the effect of the test compound on the expression of the reporter protein in the neurons, such that if there is an increase in the number of neurons expressing the reporter protein in the zebrafish contacted with the test compound compared with the number of neurons expressing the reporter protein in the zebrafish not contacted with the test compound, the compound promotes neurogenesis in the neuronally damaged zebrafish.

Further provided is a method of identifying a neuron-specific gene that is involved in neuronal function comprising: a) comparing a transgenic zebrafish expressing a reporter protein in neurons, with a transgenic zebrafish that has a neuron-specific gene knocked out or overexpressed and expresses a reporter protein in neurons; and b) determining the effect of the neuron-specific gene knockout or gene overexpression on neuronal function such that if there is a difference between the neurons of the transgenic zebrafish expressing a reporter protein in neurons and the neurons of the transgenic zebrafish that has a neuron-specific gene knocked out or overexpressed, the neuron-specific gene is involved in neuronal function.

Also provided is a method of identifying a neuron-specific gene as a target for a neuroprotective compound comprising: a) contacting a transgenic zebrafish that expresses a reporter protein in neurons and has a neuron-specific gene knocked out, with a neurotoxin and a neuroprotective compound; b) comparing the expression of the reporter protein in neurons of the transgenic zebrafish that does not have a neuron-specific gene knocked out and has been contacted with a neurotoxin and a neuroprotective compound, with the neurons of the knockout transgenic zebrafish; and c) determining the effect of the neuroprotective compound on the expression of the reporter protein in the neurons, such that if the expression of the reporter protein in the neurons of the transgenic zebrafish that does not have a neuron-specific gene knocked out is greater than the expression of the reporter protein in the knockout zebrafish, the neuron-specific gene is a target for the neuroprotective.

The present invention also provides a method of identifying a neuron-specific gene as a target for a compound that promotes neurogenesis comprising: a) contacting a neuronally damaged transgenic zebrafish expressing a reporter protein in neurons and has a neuron-specific gene knocked out with a compound that promotes neurogenesis; b) comparing the expression of the reporter protein in the neurons of the transgenic zebrafish with a neuron-specific gene knocked out with the expression of the reporter protein in the neurons of a neuronally damaged transgenic zebrafish that does not have a neuron-specific gene knocked out and has been contacted with the a compound that promotes neurogenesis; and c) determining the effect of the compound that promotes neurogenesis on the expression of the reporter protein in the neurons, such that if there is an increase in the number of neurons expressing a reporter protein in the zebrafish that does not have a neuron-specific gene knocked out compared with the number of neurons expressing a reporter protein in a transgenic zebrafish with a neuron-specific gene knocked out, the neuron-specific gene is a target for the compound that promotes neurogenesis.

The present invention further provides a method of identifying a neuron-specific gene as a target for a compound that regenerates neurons comprising: a) contacting a neuronally damaged transgenic zebrafish expressing a reporter protein in neurons and has a neuron-specific gene knocked out with a compound that regenerates neurons; b) comparing the expression of the reporter protein in the neurons of the transgenic zebrafish with a neuron-specific gene knocked out with the expression of the reporter protein in the neurons of a neuronally damaged transgenic zebrafish that does not have a neuron-specific gene knocked out and has been contacted with a compound that regenerates neurons; and c) determining the effect of the compound that regenerates neurons on the expression of the reporter protein in the neurons, such that if expression of the reporter protein in the zebrafish that does not have a neuron-specific gene knocked out is greater than the expression of the reporter protein in a transgenic zebrafish with a neuron-specific gene knocked out the neuron-specific gene is a target for the compound that regenerates neurons.

Also provided is a method of identifying a neuroprotective compound that effects neuronal protection via a neuron-specific gene comprising: a) contacting a transgenic zebrafish that expresses a reporter protein in neurons and has a neuron-specific gene knocked out with a neurotoxin and test compound; b) comparing the expression of the reporter protein in the neurons of the transgenic zebrafish with a neuron-specific gene knocked out with the expression of the reporter protein in the neurons of a neuronally damaged transgenic zebrafish that does not have a neuron-specific gene knocked out and has been contacted with the test compound; and c) determining the effect of the test compound on expression of the reporter protein in neurons, such that if expression of the reporter protein in the neurons of the zebrafish contacted with the neurotoxin and the test compound is greater than the expression of the reporter protein in the transgenic zebrafish that expresses a reporter protein in neurons and has a neuron-specific gene knocked out, the compound is a neuroprotective compound that effects neuroprotection via the neuron-specific gene that has been knocked out.

Also provided is a method of identifying a compound that regenerates neurons via a neuron-specific gene comprising: a) contacting a neuronally damaged transgenic zebrafish that expresses a reporter protein in neurons and has a neuron-specific gene knocked out with a test compound; b) comparing the expression of the reporter protein in the neurons of the transgenic zebrafish with a neuron-specific gene knocked out with the expression of the reporter protein in the neurons of a neuronally damaged transgenic zebrafish that does not have a neuron-specific gene knocked out and has been contacted with the test compound; and c) determining the effect of the test compound on expression of the reporter protein in neurons, such that if expression of the reporter protein in the neurons of the zebrafish contacted with the test compound is greater than the expression of the reporter protein in the transgenic zebrafish that expresses a reporter protein in neurons and has a neuron-specific gene knocked out, the compound is a compound that regenerates neurons via the neuron-specific gene that has been knocked out.

Also provided is a method of identifying a compound that promotes neurogenesis via a neuron-specific gene comprising: a) contacting a neuronally damaged transgenic zebrafish that expresses a reporter protein in neurons and has a neuron-specific gene knocked out with a test compound; b) comparing the expression of the reporter protein in the neurons of the transgenic zebrafish with a neuron-specific gene knocked out with the expression of the reporter protein in the neurons of a neuronally damaged transgenic zebrafish that does not have a neuron-specific gene knocked out and has been contacted with the test compound; and; c) determining the effect of the test compound on expression of the reporter protein in neurons, such that if there is an increase in the number of neurons expressing the reporter protein in the zebrafish contacted with the test compound compared with the number of neurons expressing a reporter protein in the transgenic zebrafish that expresses a reporter protein in neurons and has a neuron-specific gene knocked out, the compound promotes neurogenesis via the neuron-specific gene that has been knocked out.

The present invention also provides a method of obtaining a gene associated with neuroprotection comprising: a) mutagenizing a transgenic zebrafish that expresses a reporter protein in neurons and exhibits neuroprotection in the presence of a neurotoxin and a neuroprotectant; b) administering a neurotoxin and the neuroprotectant to the mutagenized transgenic zebrafish of a); c) comparing the expression of the reporter protein in the neurons of the transgenic zebrafish of b) with the expression of the reporter protein in the neurons of an unmutagenized transgenic zebrafish that expresses a reporter protein in neurons and exhibits neuroprotection in the presence of a neurotoxin and the neuroprotectant compound; d) determining the effect of the test compound on expression of the reporter protein in neurons, such that if there is change in neuroprotection in the mutagenized zebrafish as compared to the unmutagenized zebrafish, the mutagenized zebrafish contains a mutation in a gene associated with neuroprotection; e) mapping the mutant gene; and f) cloning the gene associated with neuroprotection.

Also provided is a method of obtaining a gene associated with regeneration comprising: a) mutagenizing a transgenic zebrafish that expresses a reporter protein in neurons and that when neuronally damaged exhibits regeneration in the presence of a compound that promotes regeneration; b) neuronally damaging the mutagenized transgenic zebrafish of a); c) administering a compound that promotes regeneration; d) comparing the expression of the reporter protein in the neurons of the transgenic zebrafish of b) with the expression of the reporter protein in the neurons of an unmutagenized transgenic zebrafish that expresses a reporter protein in neurons and that when neuronally damaged exhibits regeneration in the presence of a compound that promotes regeneration; e) determining the effect of the test compound on expression of the reporter protein in neurons, such that if there is change in regeneration in the mutagenized zebrafish as compared to the unmutagenized zebrafish, the mutagenized zebrafish contains a mutation in a gene associated with regeneration; f) mapping the mutant gene; and g) cloning the gene associated with regeneration.

Also provided is a method of obtaining a gene associated with neurogenesis comprising: a) mutagenizing a transgenic zebrafish that expresses a reporter protein in neurons and that when neuronally damaged exhibits neurogenesis in the presence of a compound that promotes neurogenesis; b) neuronally damaging the mutagenized transgenic zebrafish of a); c) administering a compound that promotes neurogenesis; d) comparing the expression of the reporter protein in the neurons of the transgenic zebrafish of c) with the expression of the reporter protein in the neurons of an unmutagenized transgenic zebrafish that expresses a reporter protein in neurons and that when neuronally damaged exhibits neurogenesis in the presence of a compound that promotes neurogenesis; e) determining the effect of the test compound on expression of the reporter protein in neurons, such that if there is change in the number of neurons expressing a reporter protein in the mutagenized zebrafish as compared to the unmutagenized zebrafish, the mutagenized zebrafish contains a mutation in a gene associated with neurogenesis; f) mapping the mutant gene; and g) cloning the gene associated with neurogenesis.

### BRIEF DESCRIPTION OF THE FIGURES

FIG. 1 shows that GFP expression driven by the neuron-specific portion of the GATA-2 promoter is observed in the neurons of brain and spinal cord (A) and in cell bodies and axons of spinal motor neurons (B).

FIG. 2 shows that 10 μg/mL (43 μM) MPTP destroys dopaminergic populations in the zebrafish embryo while coculture with deprenyl protects neurons. Whole mount in situ hybridization of a 5 day old embryo using a probe for tyrosine hydroxylase illustrates that non-dopaminergic cell populations are unchanged in MPTP-treated embryos (arrowheads), while dopaminergic populations are absent or reduced (arrows).

### DETAILED DESCRIPTION OF THE INVENTION

The present invention may be understood more readily by reference to the following detailed description of the preferred embodiments of the invention and the Example included therein.

Before the present compounds and methods are disclosed and described, it is to be understood that this invention is not limited to specific proteins or specific methods. It is also to be understood that the terminology used herein is for the purpose of describing particular embodiments only and is not intended to be limiting.

It must be noted that, as used in the specification and the appended claims, the singular forms “a,” “an,” and “the” include plural referents unless the context clearly dictates otherwise.

The present invention is more particularly described in the following examples which are intended as illustrative only since numerous modifications and variations therein will be apparent to those skilled in the art.

The present invention provides a method of identifying a compound that protects neurons comprising: a) contacting a transgenic zebrafish expressing a reporter protein in neurons with a neurotoxin and a test compound; b) comparing the expression of the reporter protein in the neurons of zebrafish contacted with the neurotoxin and the test compound with the expression of the reporter protein in the neurons of a transgenic zebrafish that was contacted only with the neurotoxin; c) determining the effect of the test compound on the expression of the reporter protein in the neurons, such that if the expression of the reporter protein in the neurons of the zebrafish contacted with the neurotoxin and the test compound is greater than the expression of the reporter protein in the zebrafish that was contacted only with the neurotoxin, the compound protects neurons from the neurotoxin.

In the methods of this invention, the neurons that can be affected by a neurotoxin or other damage can be motor neurons, catecholaminergic neurons hippocampal neurons, forebrain neurons or dopaminergic neurons, to name a few.

The transgenic zebrafish of this invention can be a transient or a stable transgenic zebrafish. The transgenic zebrafish in which the expression of a reporter protein is tissue-specific is contemplated for this invention. For example, transgenic animals that express a reporter protein at specific sites such as neurons can be produced by introducing a nucleic acid into fertilized eggs, embryonic stem cells or the germline of the animal, wherein the nucleic acid is under the control of a specific promoter which allows expression of the nucleic acid in specific types of cells (e.g., a promoter which allows expression primarily in neurons). As used herein, a protein or gene is expressed predominantly in a given tissue, cell type, cell lineage or cell, when 90% or greater of the observed expression occurs in the given tissue cell type, cell lineage or cell.

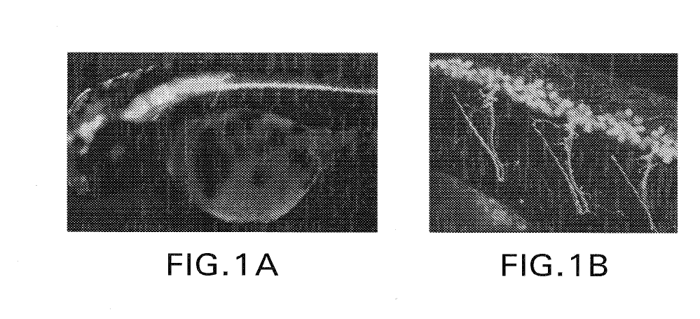

More specifically, this invention contemplates the use of a transgenic zebrafish that express a reporter protein that is under the control of the zebrafish GATA-2 promoter and is expressed in motor neurons. By utilizing a transgenic zebrafish that expresses green fluorescent protein (GFP) under the control of the neuron-specific portion of the GATA-2 promoter (Meng, et al., 1997), motor neuron cell bodies and their projecting axons are clearly visible in the spinal cord, as well as many clusters of neurons in the retina and brain (see FIG. 1). Zebrafish embryos can be easily cultured in 96 well plates where they can be soaked in solutions of THA, NMDA or any other neurotoxin. Damage to motor neurons in this assay should be readily apparent by monitoring fluorescence. Protection or repair of motor neurons by either coculturing with D-AP5 or microinjection of cDNAs encoding neurotrophic factors can also be observed.

The present invention also provides a transgenic zebrafish that expresses a reporter protein that is under the control of the zebrafish tyrosine hydroxylase promoter and is expressed in catecholaminergic and dopaminergic neurons. The promoter for the dopamine transporter gene (Holzschuh et al.) can also be used to drive dopaminergic neuron-specific expression of a reporter protein. For tissue-specific expression in all or most neurons, expression sequences for the elav or islet-2 genes can be used. The expression sequences used to drive expression of the reporter proteins can be isolated by one of skill in the art, for example, by screening a genomic zebrafish library for sequences upstream of the zebrafish gene of interest. The expression sequences can include a promoter, an enhancer, a silencer and necessary information processing sites, such as ribosome binding sites, RNA splice sites, polyadenylation sites and transcriptional terminator sequences. For example, the expression sequences can comprise neuronal promoter sequences. The expression sequences can also comprise neuronal enhancer sequences.

The transgenic fish utilized in the methods of this invention are produced by introducing a transgenic construct into cells of a zebrafish, preferably embryonic cells, and most preferably in a single cell embryo, essentially as described in Meng et al. (1998). The transgenic construct is preferably integrated into the genome of the zebrafish, however, the construct can also be constructed as an artificial chromosome. The transgenic construct can be introduced into embryonic cells using any technique known in the art. For example, microinjection, electroporation, liposomal delivery and particle gun bombardment can all be utilized to effect transgenic construct delivery to embryonic cells as well as other methods standard in the art for delivery of nucleic acids to zebrafish embryos or embryonic cells. Embryos or embryonic cells can be obtained as described in the Examples provided herein. Zebrafish containing a transgene can be identified by numerous methods such as probing the genome of the zebrafish for the presence of the transgene construct by Northern or Southern blotting. Polymerase chain reaction techniques can also be employed to detect the presence of the transgene. Expression of the reporter protein can be also be detected by methods known in the art. For example, RNA can be detected using any of numerous nucleic acid detection techniques. Alternatively, an antibody can be used to detect the expression product or one skilled in the art can visualize and quantitate expression of a fluorescent reporter protein such as GFP.

As used herein, a reporter protein is any protein that can be specifically detected when expressed. Reporter proteins are useful for detecting or quantitating expression from expression sequences. For example, operatively linking nucleotide sequences encoding a reporter protein to a tissue specific expression sequence allows one to study lineage development, such as the development of neurons. In such studies, the reporter protein serves as a marker for monitoring developmental processes, such as neuronal development, regeneration, neurogenesis and neuronal cell death. Many reporter proteins are known to one of skill in the art. These include, but are not limited to, beta-galactosidase, luciferase, and alkaline phosphatase that produce specific detectable products. Fluorescent reporter proteins can also be used, such as green fluorescent protein (GFP), cyan fluorescent protein (CFP), red fluorescent protein (RFP) and yellow fluorescent protein (YFP). For example, by utilizing GFP, fluorescence is observed upon exposure to ultraviolet light without the addition of a substrate. The use of a reporter proteins that, like GFP, are directly detectable without requiring the addition of exogenous factors are preferred for detecting or assessing gene expression during zebrafish embryonic development. Fluorescent proteins can be isolated from many different species, including but not limited to, Aequorea victoria (Chalfie, et al., 1994), Zoanthus species (Matz, et al., 1999), and Renilla reniformis (Ward and Cormier, 1979). The present invention also contemplates utilizing fluorescent reporters that have a short half life in order to monitor damage to the fluorescent neurons of the transgenic zebrafish. A transgenic zebrafish embryo, carrying a construct encoding a reporter protein and a tissue-specific expression sequence, such as an expression sequence that directs expression in neurons provides a rapid, real time in vivo system for analyzing spatial and temporal expression patterns of neuronal development, neuronal regeneration, neurogenesis and neuronal cell death.

The neurotoxins that can be utilized in the methods of this invention to effect neuronal damage include, but are not limited to, 1-methyl-4-phenyl-1,2,3,6-tetrahydropyridine (MPTP), diisopropylfluorophosphate, strychnine, kainic acid, p-chloroamphetamine, trimethylolpropane phosphate (TMPP), 6-hydroxydopamine, okadaic acid (Arendt et al., 1998), threohydroxyaspartate (THA), N-methyl-D-aspartate (NMDA), rotenone (Betarbet, et al., 2000), reserpine, methampenamine (reviewed by Tolwani et al., 1999) and an endogenous neurotoxin, NBmethyl(R)salsolinol (Naoi et al., 2000). MPTP (1-methyl-4-phenyl-1,2,3,6-tetrahydropyridine) is a neurotoxin that causes a Parkinsonian-like syndrome in humans (Langston, et al., 1984). MPTP, as well as 6-hydroxydopamine, have been shown to cause selective damage to the dopaminergic neurons affected in Parkinson's disease. Other methods of effecting neuronal damage are also contemplated by this invention, such as, but not limited to, application of a laser or physical perturbation of neurons.

Other methods of neuronal damage include overexpression or other manipulations of proteins found in plaques, neurofibrillary tangles, Hirano bodies, Pick bodies, or Lewy bodies in human neurons that are associated with human neurodegenerative disease. Examples of genes that can be overexpressed or manipulated include the genes encoding alpha synuclein (involved in Parkinsons's disease), alleles of apolipoprotein. E, presenilin, tau proteins, amyloid precursor protein (involved in Alzheimer's disease) and superoxide dismutase 1 (involved in amyotrophic lateral sclerosis). By damaging neurons in this manner, Parkinsons's disease, Alzheimers's disease, amyotrophic lateral sclerosis and other neurological disorders can be studied utilizing the methods of the present invention.

Also, by using an inducible, tissue-specific expression system, dopaminergic or other specific subsets of neurons could be damaged or destroyed by activation of a toxic protein such as diptheria toxin. This system, which utilizes a small molecule dimerizing drug to activate transcription of an exogenous target gene has been described by Pollock et al. (2000) and can be modified for use in the present methods. For example, one could drive expression of the artificial transcription factor cassette in transgenic fish with tissue-specific promoters. In a separate transgenic fish line, the gene for diptheria toxin would be placed downstream of a DNA binding domain that is recognized exclusively by the artificial transcription factor. A fish line could be generated that contains all components of the inducible system. Thus, activation of the toxin in particular groups of cells could be accomplished by placing embryos in a solution of the small molecule drug. The drug could be removed to turn off transcription of the toxin gene. Other inducible systems that can be used include the heat shock promoter (Halloran et al., 2000), a promoter that can be induced by dexamethasone (de Graaf, et al. 1998). Other systems include the tetracycline inducible system, the RU486/mifepristone inducible system and the ecdysone inducible system (reviewed by Rossi and Blau, 1998). Inducible systems can also be used to allow induction of the fluorescent protein at designated times during development, expanding the temporal specificity of fluorescent protein expression.

Embryos can be contacted with any of these neurotoxins or any other compound found to effect neuronal damage, starting at approximately 10 hours after fertilization or before differentiation of neurons begins. Alternatively, neurons can be physically perturbed or manipulated by method standard in the art and as described above. One skilled in the art would know how to select the time point for beginning administration of the neurotoxin based on the extent of neuronal differentiation. Alternatively, neurotoxin treatment can begin approximately 18 hours after fertilization or when dopaminergic neurons are detectable. Treatment can continue for several hours to several days depending on the extent of neuronal damage desired as well as a particular neurotoxin's time course for neuronal damage. The extent of neuronal damage can be assessed by detecting fluorescence, performing in situ hybridization or immunohistochemical analysis. For example, by using antibodies and/or digoxygenin-labeled RNA probes to tyrosine hydroxylase, one can determine if MPTP or other neurotoxins causes specific destruction of neurons in the zebrafish equivalent of the substantia nigra.

The test compounds used in the methods described herein can be, but are not limited to, chemicals, small molecules, drugs and secreted proteins. Test compounds that are potential neuroprotectants can be added before or concurrently with neurotoxin treatment. Test compounds that potentially cause regeneration or neurogenesis can be added after neurotoxin treatment has been discontinued. Several known neuroprotectants can be utilized as controls to determine the extent of neuroprotection by test compounds. These include, L-deprenyl and riluzole for the MPTP neurotoxin and D-amino phosphonopentanoic acid (D-AP5) for the NMDA and THA neurotoxins.

For example, one skilled in the art would select transgenic zebrafish embryos that express a reporter protein in neurons as described in the Examples. If the reporter protein is a fluorescent reporter protein, the skilled artisan will see the fluorescent reporter protein expressed in neuronal cell bodies and axons. In order to assess the protective properties of a test compound, one would contact the zebrafish with the test compound prior to addition of the neurotoxin, or contact the zebrafish with the test compound and the neurotoxin concurrently. The effects of the test compound are assessed by observing detectable changes in fluorescence, in situ hybridization signal, or immunohistochemical signal. In the absence of the test compound, the neurotoxin effects damage to neurons that can be measured both qualitatively and quantitatively. Therefore, a decrease in fluorescence is observed after addition of a neurotoxin. Thus, if a test compound is neuroprotective, upon comparison with a zebrafish exposed only to a neurotoxin, greater fluorescence is observed in the cells contacted with both the test compound and the neurotoxin. In the methods of the present invention, the transgenic zebrafish that is exposed only to the neurotoxin is also a transgenic zebrafish expressing a reporter protein in neurons.

Further provided by the present invention is a method of identifying a compound that regenerates neurons comprising: a) contacting a neuronally damaged transgenic zebrafish expressing a reporter protein in neurons with a test compound; b) comparing the expression of the reporter protein in the neurons of the zebrafish contacted with the test compound with the expression of the reporter protein in the neurons of a neuronally damaged transgenic zebrafish that was not contacted with the test compound; c) determining the effect of the test compound on the expression of the reporter protein in the neurons, such that if the expression of the reporter protein in the neurons of the zebrafish contacted with the test compound is greater than the expression of the reporter protein in the zebrafish not contacted with the test compound, the compound regenerates neurons in the neuronally damaged zebrafish.

In order to test for regeneration, a test compound is administered to the zebrafish embryo after neuronal damage has occurred. Neuronal damage can range from decreased neuronal activity to total ablation of neurons. A neuronally damaged zebrafish can be produced by administering a neurotoxin, or by obtaining a neuronally damaged zebrafish from other sources or by other means. In order to assess neuroregeneration, one skilled in the art could determine how much neuronal damage had occurred in the zebrafish by, for example, observing whether or not there is any fluorescence reporter protein production in neurons. Upon administration of the test compound, if an increase in fluorescence occurs in the previously damaged neurons, neuronal regeneration has occurred. If increased fluorescence is observed in neurons previously observed to be expressing no fluorescent reporter protein or a small amount of a fluorescent protein, the test compound is a neuroregenerative compound.

Also provided by the present invention is a method of identifying a compound that promotes neurogenesis comprising: a) contacting a neuronally damaged transgenic zebrafish expressing a reporter protein in neurons with a test compound; b) comparing the expression of the reporter protein in the neurons of zebrafish contacted with the test compound with the expression of the reporter protein in the neurons of a neuronally damaged transgenic zebrafish that was not contacted with the test compound; c) determining the effect of the test compound on the expression of the reporter protein in the neurons, such that if there is an increase in the number of neurons expressing the reporter protein in the zebrafish contacted with the test compound compared with the number of neurons expressing the reporter protein in the zebrafish not contacted with the test compound, the compound promotes neurogenesis in the neuronally damaged zebrafish.

In order to test for neurogenesis, a test compound is administered to the zebrafish embryo after neuronal damage has occurred. As used herein, neurogenesis is defined as proliferation of neurons. A neuronally damaged zebrafish can be produced by administering a neurotoxin, or by obtaining a neuronally damaged zebrafish from other sources or by other means. In order to assess neurogenesis, one skilled in the art could determine how much neuronal damage had occurred in the zebrafish by, for example, observing how many, if any neurons are expressing the fluorescent reporter protein. Upon administration of the test compound, if there is an increase in the number of neurons expressing the fluorescent protein, neurogenesis has occurred and the test compound promotes neurogenesis.

Also provided by the present invention is a method of identifying neuron-specific genes with neurological function comprising: a) constructing a zebrafish neuron cDNA library; and b) identifying a neuronal specific gene. Construction of the library is accomplished by methods standard in the art as well as those set forth in the Examples. The library can be constructed from dopaminergic neurons, motor neurons, catecholaminergic neurons or any other neurons of the transgenic zebrafish of this invention. The identification of neuron-specific genes from a library is also described in the Examples. Upon identification of neuron-specific genes, one of skill in the art would know how to compare the zebrafish sequence with other sequences in available databases in order to identify a human homologue of a neuron specific zebrafish gene. One of skill in the art would also be able to identify other homologues such as a mouse homologue or a rat homologue. Alternatively, sequences from the neuron-specific zebrafish gene can be utilized as probes to screen a human library and identify human homologs. The zebrafish sequences can also be utilized to screen other animal libraries, such as a mouse library or a rat library. Upon identification of a mouse, rat or other animal homologue, these sequences can be utilized to screen for a human homologue, either by searching available databases, or screening a human library.

Upon identification of a neuron-specific gene, the present invention also contemplates knocking out or overexpressing neuron-specific genes in zebrafish in order to determine their role in neurological function. For example, a transgenic zebrafish of the present invention that expresses a reporter protein in neurons can also have a neuron-specific gene knocked out or overexpressed. One of skill in the art would compare embryonic development of this fish with a transgenic zebrafish expressing a reporter protein in neurons that does not have the neuron-specific gene knocked out. If there is a difference in the characteristics of the neurons and their interactions, the gene that has been knocked out or overexpressed plays a role in normal neuronal function. The differences observed can be in neuronal development, neuronal regeneration, neurogenesis, neuronal cell death or any other function associated with neurons.

Thus, the present invention also provides a method of identifying a neuron-specific gene that is involved in neuronal function comprising: a) comparing a transgenic zebrafish expressing a reporter protein in neurons, with a transgenic zebrafish that has a neuron-specific gene knocked out or overexpressed and expresses a reporter protein in neurons; and b) determining the effect of the neuron-specific gene knockout or gene overexpression on neuronal function such that if there is a difference between the neurons of the transgenic zebrafish expressing a reporter protein in neurons and the neurons of the transgenic zebrafish that has a neuron-specific gene knocked out or overexpressed, the neuron-specific gene is involved in neuronal function. For example, if a neuron specific gene is knocked out in a transgenic zebrafish that normally expresses a reporter protein in neurons and the knockout results in decreased expression of the reporter protein in neurons as compared to a transgenic zebrafish that expresses a reporter protein in neurons and does not have a gene knockout, the knocked out gene is involved in neuronal function.

Also provided by the present invention is a method of identifying a neuron-specific gene as a target for a neuroprotective compound comprising: a) contacting a transgenic zebrafish that expresses a reporter protein in neurons and has a neuron-specific gene knocked out, with a neurotoxin and a neuroprotective compound; b) comparing the expression of the reporter protein in neurons of the transgenic zebrafish that does not have a neuron-specific gene knocked out and has been contacted with a neurotoxin and a neuroprotective compound, with the neurons of the knockout transgenic zebrafish; and d) determining the effect of the neuroprotective compound on the expression of the reporter protein in the neurons, such that if the expression of the reporter protein in the neurons of the transgenic zebrafish that does not have a neuron-specific gene knocked out is greater than the expression of the reporter protein in the knockout zebrafish, the neuron-specific gene is a target for the neuroprotective compound.

Also provided by the present invention is a method of identifying a neuron-specific gene as a target for a compound that promotes neurogenesis comprising: a) contacting a neuronally damaged transgenic zebrafish expressing a reporter protein in neurons and has a neuron-specific gene knocked out with a compound that promotes neurogenesis; b) comparing the expression of the reporter protein in the neurons of the transgenic zebrafish with a neuron-specific gene knocked out with the expression of the reporter protein in the neurons of a neuronally damaged transgenic zebrafish that does not have a neuron-specific gene knocked out and has been contacted with the a compound that promotes neurogenesis; and c) determining the effect of the compound that promotes neurogenesis on the expression of the reporter protein in the neurons, such that if there is an increase in the number of neurons expressing a reporter protein in the zebrafish that does not have a neuron-specific gene knocked out compared with the number of neurons expressing a reporter protein in a transgenic zebrafish with a neuron-specific gene knocked out the neuron-specific gene is a target for the compound that promotes neurogenesis.

Also provided by the present invention is a method of identifying a neuron-specific gene as a target for a compound that regenerates neurons comprising: a) contacting a neuronally damaged transgenic zebrafish expressing a reporter protein in neurons and has a neuron-specific gene knocked out with a compound that regenerates neurons; b) comparing the expression of the reporter protein in the neurons of the transgenic zebrafish with a neuron-specific gene knocked out with the expression of the reporter protein in the neurons of a neuronally damaged transgenic zebrafish that does not have a neuron-specific gene knocked out and has been contacted with a compound that regenerates neurons; and c) deter mining the effect of the compound that regenerates neurons on the expression of the reporter protein in the neurons, such that if expression of the reporter protein in the zebrafish that does not have a neuron-specific gene knocked out is greater than the expression of the reporter protein in a transgenic zebrafish with a neuron-specific gene knocked out the neuron-specific gene is a target for the compound that regenerates neurons.

The present invention also provides a method of identifying a neuroprotective compound that effects neuronal protection via a neuron-specific gene comprising: a) contacting a transgenic zebrafish that expresses a reporter protein in neurons and has a neuron-specific gene knocked out with a neurotoxin and test compound; b) comparing the expression of the reporter protein in the neurons of the transgenic zebrafish with a neuron-specific gene knocked out with the expression of the reporter protein in the neurons of transgenic zebrafish that does not have a neuron-specific gene knocked out and has been contacted with a neurotoxin and test compound; and c) determining the effect of the test compound on expression of the reporter protein in neurons, such that if expression of the reporter protein in the neurons of the transgenic zebrafish that expresses a reporter protein in neurons contacted with the neurotoxin and the test compound is greater than the expression of the reporter protein in the transgenic zebrafish that expresses a reporter protein in neurons and has a neuron-specific gene knocked out, the compound is a neuroprotective compound that effects neuroprotection via the neuron-specific gene that has been knocked out.

In one example, neuronal damage can be induced in a transgenic zebrafish expressing a reporter protein in neurons and in a transgenic fish expressing a reporter protein in neurons and containing a neuron-specific gene knockout. A test compound is then administered to both fish. Either fish can receive the test compound first. One of skill in the art would then compare the knockout zebrafish with the zebrafish expressing a reporter protein in neurons that does not have a neuron-specific gene knockout. If a decrease in expression of the reporter protein is observed in the knockout zebrafish as compared to the zebrafish that does not have a neuron-specific gene knockout, the test compound is a neuroprotective agent that affects neuronal function via the neuron-specific gene that has been knocked out. The neuroprotective agent may be involved in transcription of this gene, translation of a protein encoded by the neuron specific gene or it may interacting with the neuron-specific protein produced by this gene to effect neuroprotection.

The present invention also provides a method of identifying a compound that regenerates neurons via a neuron-specific gene comprising: a) contacting a neuronally damaged transgenic zebrafish that expresses a reporter protein in neurons and has a neuron-specific gene knocked out with a test compound; b) comparing the expression of the reporter protein in the neurons of the transgenic zebrafish with a neuron-specific gene knocked out with the expression of the reporter protein in the neurons of a neuronally damaged transgenic zebrafish that does not have a neuron-specific gene knocked out and has been contacted with the test compound; c) determining the effect of the test compound on expression of the reporter protein in neurons, such that if expression of the reporter protein in the neurons of the zebrafish contacted with the test compound is greater than the expression of the reporter protein in the transgenic zebrafish that expresses a reporter protein in neurons and has a neuron-specific gene knocked out, the compound is a compound that regenerates neurons via the neuron-specific gene that has been knocked out.

Also provided is a method of identifying a compound that promotes neurogenesis via a neuron-specific gene comprising: a) contacting a neuronally damaged transgenic zebrafish that expresses a reporter protein in neurons and has a neuron-specific gene knocked out with a test compound; b) comparing the expression of the reporter protein in the neurons of the transgenic zebrafish with a neuron-specific gene knocked out with the expression of the reporter protein in the neurons of a neuronally damaged transgenic zebrafish that does not have a neuron-specific gene knocked out and has been contacted with the test compound; c) determining the effect of the test compound on expression of the reporter protein in neurons, such that if there is an increase in the number of neurons expression of the reporter protein in the zebrafish contacted with the test compound compared with the number of neurons expressing a reporter protein in the transgenic zebrafish that expresses a reporter protein in neurons and has a neuron-specific gene knocked out, the compound promotes neurogenesis via the neuron-specific gene that has been knocked out.

Once the particular effect of a compound is determined by the methods of the present invention, the transgenic fish expressing a reporter protein in neurons can be mutagenized with ethylnitrosurea (ENU) to induce a large number of point mutations. Since the effect of a particular compound on the unmutagenized zebrafish will be known, the progeny of mutagenized fish can then contacted with the compound and evaluated for alterations in their response to the compound. Mutants that affect the response of zebrafish embryos to therapeutic compounds can then be mapped and the relevant genes cloned according to methods standard in the art. For examples, if a compound known to be a neuroprotective compound is found to be a neuroprotective compound by the methods of the present invention or by other means, the zebrafish expressing a reporter protein in neurons can be mutagenized and the progeny of these zebrafish can be contacted with the neuroprotective compound to determine if mutagenesis results in a change in the transgenic zebrafish's response to the compound. If the mutagenized zebrafish responds differently to the neuroprotective compound, the mutations can be mapped and the genes clones in order to further study the role of these genes in neuroprotection. Similar studies can be conducted with compounds that regenerate neurons as well as with compounds that promote neurogenesis.

Therefore, the present invention provides a method of obtaining a gene associated with neuroprotection comprising: a) mutagenizing a transgenic zebrafish that expresses a reporter protein in neurons and exhibits neuroprotection in the presence of a neurotoxin and a neuroprotectant; b) administering a neurotoxin and the neuroprotectant to the mutagenized transgenic zebrafish of a); c) comparing the expression of the reporter protein in the neurons of the transgenic zebrafish of b) with the expression of the reporter protein in the neurons of an unmutagenized transgenic zebrafish that expresses a reporter protein in neurons and exhibits neuroprotection in the presence of a neurotoxin and the neuroprotectant compound; d) determining the effect of the test compound on expression of the reporter protein in neurons, such that if there is change in neuroprotection in the mutagenized zebrafish as compared to the unmutagenized zebrafish, the mutagenized zebrafish contains a mutation in a gene associated with neuroprotection; e) mapping the mutant gene; and f) cloning the gene associated with neuroprotection.

Also provided is a method of obtaining a gene associated with regeneration comprising: a) mutagenizing a transgenic zebrafish that expresses a reporter protein in neurons and that when neuronally damaged exhibits regeneration in the presence of a compound that promotes regeneration; b) neuronally damaging the mutagenized transgenic zebrafish of a); c) administering a compound that promotes regeneration; d) comparing the expression of the reporter protein in the neurons of the transgenic zebrafish of b) with the expression of the reporter protein in the neurons of an unmutagenized transgenic zebrafish that expresses a reporter protein in neurons and that when neuronally damaged exhibits regeneration in the presence of a compound that promotes regeneration; e) determining the effect of the test compound on expression of the reporter protein in neurons, such that if there is change in regeneration in the mutagenized zebrafish as compared to the unmutagenized zebrafish, the mutagenized zebrafish contains a mutation in a gene associated with regeneration; f) mapping the mutant gene; and g) cloning the gene associated with regeneration.

Further provided is a method of obtaining a gene associated with neurogenesis comprising: a) mutagenizing a transgenic zebrafish that expresses a reporter protein in neurons and that when neuronally damaged exhibits neurogenesis in the presence of a compound that promotes neurogenesis; b) neuronally damaging the mutagenized transgenic zebrafish of a); c) administering a compound that promotes neurogenesis; d) comparing the expression of the reporter protein in the neurons of the transgenic zebrafish of c) with the expression of the reporter protein in the neurons of an unmutagenized transgenic zebrafish that expresses a reporter protein in neurons and that when neuronally damaged exhibits neurogenesis in the presence of a compound that promotes neurogenesis; e) determining the effect of the test compound on expression of the reporter protein in neurons, such that if there is change in the number of neurons expressing a reporter protein in the mutagenized zebrafish as compared to the unmutagenized zebrafish, the mutagenized zebrafish contains a mutation in a gene associated with neurogenesis; f) mapping the mutant gene; and g) cloning the gene associated with neurogenesis.

The present invention is more particularly described in the following examples which are intended as illustrative only since numerous modifications and variations therein will be apparent to those skilled in the art.

### EXAMPLES

This invention allows characterization of the function of secreted proteins, small molecules and other compounds in zebrafish (Danio rerio) embryos with the ultimate aim of identifying new therapies for human diseases. This protocol encompasses the creation and use of transgenic zebrafish strains that express fluorescent proteins in specific subsets of neurons. Embryos are soaked in solutions of small molecules to determine their effect. These studies provide a way to rapidly screen thousands of proteins, small molecules and other compounds for function and toxicity in a whole vertebrate organism. In addition, new transgenic lines will be produced by injecting embryos with plasmid DNA constructs and raising them to adulthood.

Zebrafish are placed in mating cages the night before an experiment. The next morning, eggs are collected from the bottom of the cages and treated with pronase to remove the chorions. Plasmid DNA constructs or RNA are injected into embryos at the 1-4 cell stage (˜30 minutes after fertilization). Embryos are monitored for the next few days under a fluorescence microscope. Some embryos are raised to adulthood.

For small molecule treatments, embryos are soaked in chemicals or drugs of interest. For example, embryos are soaked in neurotoxic chemicals to destroy neurons that develop between 1-2 days after fertilization. Following this treatment, embryos are treated with potentially therapeutic small molecules. For initial experiments, embryos are anesthetized (see below) and fixed in 4% paraformaldehyde for in situ hybridization and immunohistochemistry.

Zebrafish embryos have been shown to be an extremely useful model to study vertebrate development. The embryos are transparent, are produced in large numbers and develop outside of the mother. Strains of transgenic zebrafish that express the fluorescent proteins in organs that are affected in neurodegenerative diseases, i.e. neurons are provided by this invention. The transparency of the embryos allows observation of fluorescent cells and evaluation of the effect of chemicals and drugs on them.

The zebrafish are housed in a state-of-the-art recirculating aquaculture system that provides constant aeration, filtration and UV sterilization of water. They are fed twice a day with a combination of live baby brine shrimp and dry powdered food. Any fish that appear to be ill will be removed and euthanized, as described below. Alternatively, tanks of sick of fish will be treated with nitrofurazone.

To avoid possible discomfort to older stage embryos, they are immersed in a solution of 0.016% tricaine (3-amino benzoic acidethylester, also known as MS-222). This method will also be used if invasive procedures are required for adult fish or if fish or embryos need to be euthanized.

### Manipulations

Mating pairs of adult zebrafish are placed in mating cages in the evening. The following morning, eggs are collected from the bottom of the mating cage and fertile embryos are placed in fresh fish water. At the end of the day, (embryos should be at the late gastrulation stage) phenylthiourea (PTU, Sigma-Aldrich) is added to a final concentration of 0.003% to prevent formation of pigment. At approximately 24 hours past fertilization (hpf), embryos are transferred to a 24 or 96 well plate for treatment in different concentrations of neurotoxins. Zebrafish embryos at this stage in development have completed much of their brain development and primary neurogenesis (Kimmel, et al., 1995). Motor neurons and some dopaminergic neurons are detectable at this stage (Eisen, et al., 1986; Guo, et al., 1999).

Initial concentrations of neurotoxins, including MPTP, hydroxydopamine, NMDA and THA (all purchased from Sigma-Aldrich), are determined based on the concentrations administered to goldfish (Goping, et al., 1995) and that used in cultured spinal cord explant experiments (Rothstein, et al., 1993; Annis and Vaugn, 1998). One of skill can adjust the concentrations depending on the level of neuronal damage desired and the time frame for neuronal damage.

Since MPTP is known to cause Parkinson's disease in humans, appropriate precautions are taken to protect workers. All manipulations with MPTP are performed under a chemical fume hood and scientists wear appropriate protective clothing. Waste solutions of MPTP are detoxified by permanganate oxidation (Yang, et al., 1988). Other neurotoxins will be handled with similar care.

After embryos are secured in plates with lids, plates are transferred to an incubator, preset to 28.5° C., the optimal temperature for zebrafish development. At different time points (1, 2, and 3 days after treatment), untreated embryos and embryos treated with MPTP or hydroxydopamine are fixed in 4% paraformaldehyde and prepared for in situ hybridization and/or immunohistochemistry.

### Analyses

In situ hybridization is performed as described by Thisse, et al. (1993). A plasmid containing a partial cDNA sequence encoding tyrosine hydroxylase was obtained from A. Rosenthal (Guo, et al., 1999). An antisense RNA probe to this gene can be synthesized by in vitro transcription, incorporating digoxygenin-labeled UTP (Roche Molecular Biochemicals). Embryos are hybridized with probe overnight at 70 degrees in 50% formamide buffer. After several washes, embryos are incubated overnight with an antibody to digoxygenin conjugated to alkaline phosphatase (Roche Molecular Biochemicals). After several additional washes, embryos are developed in an alkaline solution containing nitro blue tetrazolium (NBT) and 5-bromo-4-chloro-3-indolyl phosphate (BCIP).

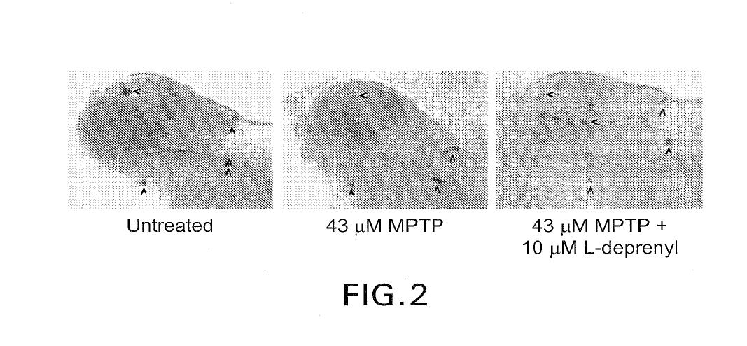

Utilizing the methods described herein, this invention shows that dopaminergic neurons in zebrafish embryos treated with MPTP (10 μg/mL or about 43 μM) are significantly reduced. As shown in FIG. 2, dopaminergic neurons are more sensitive to MPTP than other catecholaminergic neurons. MPTP-induced damage can be prevented by coincubation of embryos with L-deprenyl, a compound that inhibits the conversion of MPTP to its toxic metabolite, MPP+ (Heikkila, et al., 1984). L-deprenyl, also known as selegiline, is currently marketed by Somerset Pharmaceuticals under the trade name Eldepryl. Along with L-Dopa, L-deprenyl is one of only two drugs approved for the treatment of Parkinson's disease. Thus, this invention shows that this assay can be utilized to identify compounds with specific neuroprotective activity.

Immunohistochemistry is performed essentially as described by Jowett (1999). Embryos are incubated over night in a polyclonal antibody to tyrosine hydroxylase (Chemicon). After several washes, embryos are incubated in a biotinylated secondary antibody and finally in an avidin and biotinylated horseradish peroxidase macromolecular complex (Vector Laboratories). Final color development is achieved with diaminobenzidine (DAB). DAB is a known carcinogen and appropriate precautions will be taken for handling and disposal.

Embryos that express GFP specifically in neurons (under control of the neuron-specific portion of the GATA-2 promoter) are treated with NMDA or TEA and observed directly under a fluorescence microscope. Effects of neurotoxins on embryos are determined both qualitatively and quantitatively (by counting motor neuron cell bodies).

### Neuroprotective Compounds

This study is performed essentially as described above, except that neuroprotective drugs (levodopa, deprenyl, and D-AP5, Sigma-Aldrich) are added to the embryo cultures at selected time points, both concurrently with neurotoxins and after neurotoxins have exerted their effects. Initial concentrations are determined based on that used in goldfish and tissue culture experiments. Again, results are measured by examination of neurons in both a qualitative and quantitative manner, as described above.

### Transgenic Zebrafish Line Expressing GFP in Dopaminergic Neurons

### Promoter Identification

The promoter for tyrosine hydroxylase can be isolated and used to create a new line of transgenic zebrafish that will express a fluorescent protein in catecholaminergic neurons. Although this line should express fluorescence in noradrenergic neurons as well as dopaminergic neurons, the dopaminergic neurons can be easily distinguished based on their location in the ventral midbrain (Guo, et al, 1999). Primers matching the gene sequence of tyrosine hydroxylase (GenBank Accession #AF075384) were synthesized (Sigma Genosys) and shown to reliably amplify a fragment of approximately the expected size from genomic DNA. Primers were designed to account for introns in the genomic DNA. Zebrafish intron/exon boundaries were determined by comparing the zebrafish tyrosine hydroxylase gene sequence to the mouse gene sequence, in which the intron/exon boundaries are known (Iwata, et al., 1992). Experience has shown that intron position is conserved across vertebrate species. These primers were used to screen pools of DNA from a P1-derived Artificial Chromosome (PAC) library (Genome Systems, Inc.) by the polymerase chain reaction (PCR). When a positive pool was found, it was further subdivided and screened again until the number of clones in a pool was small enough to screen individual PAC clones by PCR. This procedure can also be utilized to isolate and characterize the promoter for the dopamine transporter gene.

To identify small (4-10 kb) DNA fragments that contain the promoter of interest, positive PAC clone DNA will be isolated using a DNA extraction kit (Qiagen), with a modified protocol suitable for large, low-copy number plasmids. The DNA is then restriction digested, electrophoresed and blotted to a nylon membrane (Osmonics, Inc.) for Southern hybridization to the original tyrosine hydroxylase cDNA probe (Guo, et al., 1999). A ³²P radioactive probe is made by random prime labeling (Stratagene). Standard protocols for Southern hybridization are followed (Sambrook, et al., 1989).

Guo, et al. (1999), described a class of neurons in the zebrafish that express tyrosine hydroxylase, the enzyme that catalyzes conversion of tyrosine to dopa and is expressed specifically in catecholaminergic neurons. Using whole mount in situ hybridization and immunohistochemistry, they showed that the zebrafish brain contains a class of neurons that can be considered dopaminergic, since they contain the enzymes required for synthesis of dopamine, but not the enzymes required to convert dopamine to noradrenaline and adrenaline.

To determine whether these dopaminergic neurons are sensitive to MPTP, zebrafish embryos are soaked in the chemical using prescribed dosing regimens and determine by in situ hybridization and immunohistochemistry whether the tyrosine-hydroxylase positive neurons are damaged or missing.

Although the neuron-specific GFP transgenic fish (described below) may express GFP in dopaminergic neurons, many other groups of neurons in the brain may express GFP.

### Transgenic Fish Production

Transgenic fish are produced essentially as described in Meng, et al. (1998), with minor modifications. Hybridizing fragments between the sizes of 4 to 10 kb (identified as described above) are fused to a vector containing the sequence for a green fluorescent protein (Clontech or Stratagene). Fifty picograms of this DNA construct (in 1 mL containing 0.2% phenol red) are injected into zebrafish embryos at the one cell stage using a PLI 100 pressure injector (Harvard Apparatus). Embryos are cultured in Holtfreter's solution (60 mM NaCl, 2.4 mM sodium bicarbonate, 0.8 mM calcium chloride, 0.67 mM potassium chloride) containing penicillin and streptomycin. Microscopic examination of embryos for the presence of phenol red allows separation of embryos that received the DNA construct from those that did not.

When embryos reach the age of about 30 hours, they are examined under a fluorescence microscope to determine whether any transient expression of the green fluorescent protein is visible in the brain. Injected embryos are then raised to adulthood and screened for stable transmission of the transgene. Screening entails mating potential carriers to each other or to wild type fish and examining their offspring for appropriate fluorescence. Alternatively, a portion of the dorsal fin of potential carriers can be clipped, DNA extracted, and transgene transmission determined by PCR.

Observation of transient fluorescent protein expression in the region of the ventral midbrain after initial injection of the construct is expected. If fluorescence is detected as expected in cells of this region, the likelihood of developing a stable line is high, after enough embryos have been raised and screened in future generations. Therefore, the present invention provides for the development of stable zebrafish cell lines.

The present invention also contemplates alternative strategies for transgenic fish production. For example, a bacterial artificial chromosome (BAC) clone containing the tyrosine hydroxylase gene or the dopamine transporter gene can be isolated using techniques described above. The BAC clone can then be modified by Chi-stimulated homologous recombination to incorporate the GFP reporter gene, as described by Jessen, et al. (1998).

### Isolation of Fluorescent Neuronal Cells

Embryos produced by the mating of transgenic males and females are dechorionated in pronase solution, washed and disrupted in Holtfreter's solution (60 mM NaCl, 2.4 mM sodium bicarbonate, 0.8 mM calcium chloride, 0.67 mM potassium chloride) using a 1.5 ml pellet pestle (Kontos Glass, OEM749521-1590). After digestion with 1× Trypsin/EDTA for 15 minutes at 32° C., the cells are washed twice with phosphate buffered saline (PBS) and passed through a 40 micron nylon mesh filter. Cells are recovered by centrifugation at 400 g for 5 minutes in a Beckman tabletop centrifuge. Brains dissected from adult transgenic fish can also be used as a tissue source. Fluoresence activated cell-sorting (FACS) is performed using a standard protocol for isolating fluorescein-labeled cells. Usually, about 2% of cells from transgenic embryos are fluorescent. This will vary depending on the cell type that is fluorescent. For the final sorting, fluorescent cells are sorted directly into a buffer containing guanidinium isothiocyanate and stored at −70° C. until use. By utilizing FACS, dopaminergic or other neuron-specific cells can be purified to build a tissue-specific cDNA library with rare transcripts represented. Screening of this library by methods standard in the art can yield many new tissue-specific genes with neurological function. The following describes possible methods for RNA isolation and cDNA library construction and is not meant to exclude other methods for this step.

RNA isolation

Total RNA is extracted from FACS-purified cells using the TRIzol RNA Isolation Kit (LIFE TECHNOLOGIES, Grand Island, N.Y.) and mRNA is isolated from the total RNA using PolyATtract System 1000 (Promega, Madison, Wis.). The protocols provided by LIFE TECHNOLOGIES and Promega are utilized for isolation of mRNA. At least 50 ng of mRNA will be prepared for cDNA library construction.

cDNA Library Construction

The SMART cDNA Library Construction Kit (Clontech), which was developed for constructing high-quality cDNA libraries from small quantities of RNA is utilized. As discussed above, although either total or poly A+ RNA may be used as a template for SMART cDNA synthesis, mRNA is utilized for the purposes of the present invention.

First-Strand cDNA is synthesized using 25 ng polyA+ mRNA isolated from GFP-positive cells. SMART/5′ oligonucleotide III and CDS/3′ oligonucleotide 111 is used in the MMLV reverse transcriptase reaction. The SMART/5′ oligonucleotide III contains an Sfi I site with AAT whereas the CDS/3′ oligonucleotide III contains an Sfi I site with GGC. This variation of AAT and GGC is used because Sfi I recognizes 5′GGCCNNNNNGGCC3′.

Low cycle, long-distance PCR (LD-PCR) is used to amplify the first-strand cDNA. KlenTaq Polymerase, a new 5′ PCR primer complementary to the SMART/5′ oligonucleotide III, and the CDS/3′ oligonucleotide III are used in the reaction. Currently, it is possible to amplify enough PCR products for library construction after 10 cycles. After amplification, a sample of the PCR product is analyzed with 1-kb DNA ladder size markers to determine the size and amount of PCR product.

As mentioned above, SMART oligonucleotide III and CDS oligonucleotide III contain Sfi I restriction sites. PCR products are digested with Sfi I restriction enzyme. This digestion generates DNA fragments with 5′ AAT and 3′ GGC overhangs. Digested products are then size-fractionated. Two cDNA pools are collected: one is 1-2 kb and another one is larger than 2 kb. After purification, the size-fractionated, SE I-digested cDNA is ligated to the dephosphorylated and Sfi I digested lambda TriplEx vector. One of these arms has a Sfi I site with TTA whereas the other one has a Sfi with CCG. Therefore, the cDNA inserts are cloned into the phage aims with their 5′ ends at the TTA arms and the 3′ ends at the CCG arms. The ligated products are packaged and a small portion of it plated out on LB plates for titering. 1-2×10⁶ independent clones are usually obtained. If the titer is as expected, remaining phages are converted into plasmid, to simplify sequencing and subtraction, as described below.

### Library Characterization

First, 1,000 random clones from the library are sequences. This provides insight into the quality of the library, including the level of redundancy. Plasmid DNA obtained from the first 1,000 clones are used as driver to subtract redundant clones from the rest of the library. Normalization and subtraction is done according to Bonaldo, et al. (Bonaldo, et al., 1996). Clones are sequenced until it is decided that all potential expressed sequences from the neuron specific library have been identified. Once a neuron-specific library is produced, microarrays can be made using the genes present in the neuron-specific libraries. These microarrays can be used, for example, to examine gene expression changes that result from MPTP or other neurotoxin application. Similarly, these microarrays can be utilized to examine changes in gene expression as a result of any other neuronal damage, such as the application of a laser of physical perturbation of neurons.

To determine whether novel zebrafish genes are appropriate drug targets, bioinformatics is utilized to establish whether human homologues exist. Second, whole mount in situ hybridization is performed to establish neuronal specificity. Finally, functional information is obtained by knock-down/knock-out technology, such as morpholinos (Nasevicius and Ekker, 2000). Transient over-expression and over-expression of dominant negative constructs, when appropriate, is also used to provide functional information. Phenotypes resulting from these experiments will be examined in transgenic fish to determine the effects of knock out or overexpression on a cell type of interest. These methods can also be utilized to analyze and validate target genes potentially involved in neurological function obtained from other sources.

### High Throughput Screening

Several companies such as Imaging Research Incorporated of St. Catharines, Ontario and Union Biometrica, Inc. of Somerville, Mass. make fluorescent imagers/cell sorters designed for quantification of fluorescence in a three-dimensional organism. These imagers or any other imager, such as those produced by Perkin Elmer, sensitive enough to detect differences in fluorescence that result from neurotoxin or neuroprotective molecule application can be utilized to establish a high throughput screen. In this high throughput screening method, embryos are arrayed in 96 well plates in water or solutions of neurotoxins and/or drugs, as described above. At set time points, embryos will be collected and scanned for fluorescence.

In addition to visualizing whole embryos, embryos can be sonicated to break up into clumps of cells which will settle to the bottom of the culture plate. Thus, a traditional fluorescent plate scanner could be used to monitor fluorescence. Another possibility is to use suction to draw embryos onto a wet nitrocellulose filter and quantify fluorescence by scanning with a phosphoimager, such as the Storm phosphoimager.

An alternative strategy would involve examination of adult zebrafish neurons. Although the adult fish is no longer transparent, the relevant neurons are expected continue to express GFP. Damage can be induced in adult neurons by injection of neurotoxins and fluorescence measured by whole tissue collection and cell sorting. The effect of administration of therapeutic drugs can be measured in a similar way.

The test compounds found to effect neuronal protection, regeneration and/or neurogenesis utilizing the methods of this invention can be screened for neuronal protection, regeneration and/or neurogenesis in other animal models and/or cellular assays to determine their effectiveness as a therapeutic drug.

Throughout this application, various publications are referenced. The disclosures of these publications in their entireties are hereby incorporated by reference into this application in order to more fully describe the state of the art to which this invention pertains.

### REFERENCES

Ackerley S, Grierson A J, Brownlees I, Thornhill P, Anderton B H, Leigh P N, Shaw C E, and Miller C J. (2000). Glutamate slows axonal transport of neurofilaments in transfected neurons. J. Cell Biol. 150, 165-175.
Adam A, Bartfai R, Lele Z, Krone P H, Orban L. (2000). Heat-inducible expression of a reporter gene detected by transient assay in zebrafish. Exp Cell Res. 256(1): 282-90.
Annis C M and Vaughn J E (1998). Differential vulnerability of autonomic and somatic motor neurons to N-methyl-D-aspartate-induced excitotoxicity. Neuroscience 83, 239-249.
Arendt T, Holzer M, Bruckner M K, Janke C, and Gartner U. (1998). The use of okadaic acid in vivo and the induction of molecular changes typical for Alzheimer's Disease. Neuroscience 85, 1337-1340.
Betarbet R, Sherer T B, MacKenzie G, Garcia-Osuna M, Panov A V, and Greenamyre J T. (2000). Chronic systemic pesticide exposure reproduces features of Parkinson's disease. Nat. Neurosci. 3: 1301-1306.
Brown R H. (1997). Mitochondria and oxidative stress in amyotrophic lateral sclerosis. In Beal M F, Howell N, and Bódis-Wollner I. (eds) Mitochondria & Free Radicals in Neurodegenerative Diseases. New York: Wiley-Liss, Inc., pp. 309-318.
Björklund A and Lindvall O. (2000). Cell replacement therapies for central nervous system disorders. Nat. Neurosci. 3, 537-544.
Chalfie M, Tu Y, Euskirchen G, Ward W W, Prasher D C. (1994). Green fluorescent protein as a marker for gene expression. Science 263, 802-5.
Cleveland D W (1999). From Charcot to SOD1: Mechanisms of selective motor neuron death in ALS. Neuron 24, 515-520.
Corse A M, Bilak M M, Bilak S R, Lehar M, Rothstein J D and Kuncl R W. (1999). Preclinical testing of neuroprotective factors in a model of chronic motor neuron degeneration. Neurobiol. Dis. 6, 335-346.
de Graaf M, Zivkovic D, Joore J. (1998). Hormone-inducible expression of secreted factors in zebrafish embryos. Dev Growth Differ. 40(6): 577-82.
Dodd A, Curtis P M, Williams L C, and Love D R. (2000). Zebrafish: bridging the gap between development and disease. Hum. Mol. Genet. 9, 2443-2449.
Dooley K and Zon L I. (2000). Zebrafish: a model system for the study of human disease. Curr. Opin. Genet. Dev. 10, 252-256.
Eisen J S, Myers P Z and Westerfield M. (1986). Pathway selection by growth cones of identified motoneurons in live zebrafish embryos. Nature 320, 269-271.
Elliot J L (1999). Experimental models of amyotrophic lateral sclerosis. Neurobiol. Dis. 6, 310-320.
Goping G, Pollard H B, Adeyemo O M, and Kuijpers G A. (1995). Effect of MPTP on dopaminergic neurons in the goldfish brain: a light and electron microscope study. Brain Res. 687, 35-52.
Grünblatt E, Mandel S, and Youdim M B H. (2000). MPTP and 6-hydroxydopamine-induced neurodegeneration as models for Parkinson's disease: neuroprotective strategies. J. Neurol. 247 [Suppl 2], 95-102.
Guo S, Wilson S W, Cooke S, Chitnis A B, Driever W and Rosenthal A (1999). Mutations in the zebrafish unmask shared regulatory pathways controlling the development of catecholaminergic neurons. Dev. Biol. 208, 473-487.
Halloran M C, Sato-Maeda M, Warren J T, Su F, Lele Z, Krone P H, Kuwada J Y, Shoji W. (2000). Laser-induced gene expression in specific cells of transgenic zebrafish. Development 127(9): 1953-60.
Heikkila R E, Manzino L, Cabbat F S, Duvoisin R C. (1984). Protection against the dopaminergic neurotoxicity of 1-methyl-4-phenyl-1,2,3,6-tetrahydropyridine by monoamine oxidase inhibitors. Nature 311: 467-469.
Horner P J and Gage F H (2000). Regenerating the damaged central nervous system. Nature 407, 963-970.
Holzschuh J, Soojin R, Aberger F, Driever W. (2001). Dopamine transporter expression distinguishes dopaminergic neurons from other catecholaminergic neurons in the developing zebrafish embryo. Mech. Dev. 101: 237-243.
Hurko O and Walsh F S. (2000). Novel drug development for amyotrophic lateral sclerosis. J. Neurol. Sci. 180, 21-28.
Iwata N, Kobayashi K, Sasaoka T, Hidaka H, and Nagatsu T. (1992). Structure of the mouse tyrosine hydroxylase gene. Biochem. Biophys. Res. Commun. 182, 348-354.
Jessen J R, Meng A, McFarlane R J, Paw B H, Zon L I, Smith G R, and Lin S. (1998). Modification of bacterial artificial chromosomes through Chi-stimulated homologous recombination and its application in zebrafish transgenesis. Proc. Natl. Acad. Sci. USA 95, 5121-5126.
Jessen, J R., Willett, C E. and Lin, S. (1999). Artificial chromosome transgenesis reveals long-distance negative regulation of rag1 in zebrafish. Nature Genetics 23(1):15-16.
Jowett T. (1999). Analysis of protein and gene expression. Methods Cell Biol. 59, 63-85.
Joyce J N, Frohna P A, and Neal-Beliveau S. (1996). Functional and molecular differentiation of the dopamine system induced by neonatal denervation. Neurosci. Biobehay. Rev. 20, 453-486.
Kimmel, C B, Ballard W W, Kimmel S R, Ullmann B and Schilling T F. (1995). Stages of embryonic development of the zebrafish. Dev. Dyn. 203, 253-310.
Kordower J H, Emborg M E, Bloch J, Ma S Y, Chu Y, Leventhal L, McBride J, Chen E-Y, Palfi S, Roitberg B Z, Brown W D, Holden J E, Pyzaiski R, Taylor M D, Carvey P, Ling Z, Trono D, Hantraye P, Deglon N, and Aebishcer P. (2000). Neurodegeneration prevented by lentiviral vector delivery of GDNF in primate models of Parkinson's disease. Science 290, 767-772.
Langston J W and Ballard P (1983). Chronic parkinsonism in humans due to a product of meperidine-analog synthesis. Science 21x, 979-980.
Louvel E, Hugon J and Doble A. (1997). Therapeutic advances in amyotrophic lateral sclerosis. Trends Pharm. Sci. 18, 196-203.
Mari Z and Bódis-Wollner I. (1997), MPTP-induced parkinsonian syndrome in humans and animals: How good is the model? In Beal M F, Howell N, and Bódis-Wollner I. (eds) Mitochondria & Free Radicals in Neurodegenerative Diseases. New York: Wiley-Liss, Inc., pp. 189-228.
Matz M V, Fradkov A F, Labas Y A, Savitsky A P, Zaraisky A G, Markelov M L, and Lukyanov S A. (1999). Fluorescent proteins from nonbioluminescent Anthozoa species. Nat. Biotech. 17, 969-973.
Meng, A, Tang H, Ong B A, Farrell M J and Lin S. (1997). Promoter analysis in living zebrafish embryos identifies a cis-acting motif required for neuronal expression of GATA-2. Proc. Natl. Acad. Sci. USA 94, 6267-6272.
Meng, A M., Jessen, JH. and Lin, S. (1998). Transgenesis. Methods in Cell Biology: Zebrafish. Edited by Detrich, W., Zon, L I and Westerfield, M. Academic Press, pp. 133-148.
Mitsumoto H and Tsuzaka K. (1999). Neurotrophic factors and neuromuscular disease: I. General comments, the neurotrophin family, and neuropoietic cytokines. Muscle Nerve 22, 983-999.
Naoi M, Maruyama W, Akao Y, Zhang I, Parvez H. (2000). Apoptosis induced by an endogenous neurotoxin, N-methyl(R)salsolinol, in dopamine neurons. Toxicology 153: 123-141.
Nasevicius A, Ekker S C. (2000). Effective targeted gene ‘knockdown’ in zebrafish. Nat Genet. 26: 216-20.
Peterson R T, Link B A, Dowling J E and Schreiber S L. (2000). Small molecule developmental screens reveal the logic and timing of vertebrate development. Proc. Natl. Acad. Sci. USA 97, 12965-12969.
Pollock R, Issner R, Zoller K, Natesan S, Rivera V M, Clackson T. (2000). Delivery of a stringent dimerizer-regulated gene expression system in a single retroviral vector. Proc Natl Acad Sci USA 97: 13221-6.
Pollard H B, Kuijpers G A, Adeyemo O M, Youdim M B H and Goping G. (1996). The MPTP-induced Parkinsonian syndrome in the goldfish is associated with major cell destruction in the forebrain and subtle changes in the optic tectum. Exp. Neurol. 142, 170-178.
Rossi F M V and Blau H M. 1998. Recent advances in inducible gene expression systems. Curr. Opin. Biotech. 9: 451-456.
Sambrook, J., Fritsch, E. F., and Maniatis, T. (1989). Molecular Cloning: A Laboratory Manual. (Cold Spring Harbor, N.Y.: Cold Spring Harbor Laboratory Press).
Shaw P J and Eggett C J. (2000). Molecular factors underlying selective vulnerability of motor neurons to neurodegeneration in amyotrophic lateral sclerosis. J. Neurol. 247 [Suppl. 1], 17-27.
Silani V, Braga M, Ciammola A, Cardin V and Scarlato G. (2000). Motor neurones in culture as a model to study ALS. J. Neurol. 247 [Suppl 1], 28-36.
Steiner J P, Hamilton G S, Ross D T, Valentine H L, Guo H, Connolly M A, Liang S, Ramsey C, Li J J, Huang W, Howorth, Soni R, Fuller M, Sauer H, Nowotnik A C and Suzdak P D. (1997). Neurotrophic immunophilin ligands stimulate structural and functional recovery in neurodegenerative animal models.
Thisse C, Thisse B, Schilling T F, Postlethwait J R. (1993). Structure of the zebrafish snaill gene and its expression in wild-type, spadetail and no tail mutant embryos. Development 119, 1203-1215.
Tolwani R J, Jakowce M W, Petzinger G M, Green S, Waggie K. (1999). Experimental models of Parkinson's disease: insights from many models. Lab Anim Sci 49: 363-371.
Ward W W and Cormier M J. (1979). An energy transfer protein in coelenterate bioluminescence: characterization of the Renilla green-fluorescent protein. J. Biol. Chem. 254, 781-788.
Wong P C, Rothstein J D, and Price D L. (1998). The genetic and molecular mechanisms of motor neuron disease. Curr. Opin. Neurobiol. 8, 791-799.
Yang S C, Markey S P, Bankiewicz K S, London W T, and Lunn G. (1988). Recommended safe practices for using the neurotoxin MPTP in animal experiments. Lab Anim Sci. 38, 563-7.
Zhang Y, Dawson V L, and Dawson T M. (2000). Oxidative stress and genetics in the pathogenesis of Parkinson's disease. Neurobiol. Dis. 7, 240-250.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method of identifying a compound that protects neurons comprising:
a) contacting a transgenic zebrafish expressing a reporter protein in neurons with a neurotoxin and a test compound;
b) comparing the expression of the reporter protein in the neurons of zebrafish contacted with the neurotoxin and the test compound with the expression of the reporter protein in the neurons of a transgenic zebrafish that was contacted only with the neurotoxin;
c) determining the effect of the test compound on the expression of the reporter protein in the neurons, such that if the expression of the reporter protein in the neurons of the zebrafish contacted with the neurotoxin and the test compound is greater than the expression of the reporter protein in the zebrafish that was contacted only with the neurotoxin, the compound protects neurons from the neurotoxin.

2. The method of claim 1, wherein the neurons are motor neurons.

3. The method of claim 1, wherein the neurons are catecholaminergic neurons.

4. The method of claim 3, wherein the catecholaminergic neurons are dopaminergic neurons.

5. A method of identifying a compound that regenerates neurons comprising:
a) contacting a neuronally damaged transgenic zebrafish expressing a reporter protein in neurons with a test compound;
b) comparing the expression of the reporter protein in the neurons of the zebrafish contacted with the test compound with the expression of the reporter protein in the neurons of a neuronally damaged transgenic zebrafish that was not contacted with the test compound;
c) determining the effect of the test compound on the expression of the reporter protein in the neurons, such that if the expression of the reporter protein in the neurons of the zebrafish contacted with the test compound is greater than the expression of the reporter protein in the zebrafish not contacted with the test compound, the compound regenerates neurons in the neuronally damaged zebrafish.

6-8. (canceled)

9. A method of identifying a compound that promotes neurogenesis comprising:
a) contacting a neuronally damaged transgenic zebrafish expressing a reporter protein in neurons with a test compound;
b) comparing the expression of the reporter protein in the neurons of zebrafish contacted with the test compound with the expression of the reporter protein in the neurons of a neuronally damaged transgenic zebrafish that was not contacted with the test compound;
c) determining the effect of the test compound on the expression of the reporter protein in the neurons, such that if there is an increase in the number of neurons expressing the reporter protein in the zebrafish contacted with the test compound compared with the number of neurons expressing the reporter protein in the zebrafish not contacted with the test compound, the compound promotes neurogenesis in the neuronally damaged zebrafish.

10-12. (canceled)

13. The method of claim 1, wherein expression of the reporter protein is controlled by GATA-2 expression sequences.

14. The method of claim 1, wherein expression of the reporter protein is controlled by tyrosine hydroxylase expression sequences.

15. The method of claim 1, wherein expression of the reporter protein is controlled by dopamine transporter gene expression sequences.

16. The method of claim 1, wherein the reporter protein is expressed predominantly in neurons.

17. The method of claim 15, wherein the reporter protein is expressed predominantly in dopaminergic neurons.

18. A method of identifying a neuron-specific gene that is involved in neuronal function comprising:
a) comparing a transgenic zebrafish expressing a reporter protein in neurons, with a transgenic zebrafish that has a neuron-specific gene knocked out or overexpressed and expresses a reporter protein in neurons; and
b) determining the effect of the neuron-specific gene knockout or overexpression on neuronal function such that if there is a difference between the neurons of the transgenic zebrafish expressing a reporter protein in neurons and the neurons of the transgenic zebrafish that has a neuron-specific gene knocked out or overexpressed, the neuron-specific gene is involved in neuronal function.

19-21. (canceled)

22. A method of identifying a neuron-specific gene as a target for a neuroprotective compound comprising:
a) contacting a transgenic zebrafish that expresses a reporter protein in neurons and has a neuron-specific gene knocked out, with a neurotoxin and a neuroprotective compound;
b) comparing the expression of the reporter protein in neurons of the transgenic zebrafish that does not have a neuron-specific gene knocked out and has been contacted with a neurotoxin and a neuroprotective compound, with the neurons of the knockout transgenic zebrafish; and
c) determining the effect of the neuroprotective compound on the expression of the reporter protein in the neurons, such that if the expression of the reporter protein in the neurons of the transgenic zebrafish that does not have a neuron-specific gene knocked out is greater than the expression of the reporter protein in the knockout zebrafish, the neuron-specific gene is a target for the neuroprotective compound.

23-25. (canceled)

26. A method of identifying a neuron-specific gene as a target for a compound that promotes neurogenesis comprising:
a) contacting a neuronally damaged transgenic zebrafish expressing a reporter protein in neurons that has a neuron-specific gene knocked out with a compound that promotes neurogenesis;
b) comparing the expression of the reporter protein in the neurons of the transgenic zebrafish with a neuron-specific gene knocked out with the expression of the reporter protein in the neurons of a neuronally damaged transgenic zebrafish that does not have a neuron-specific gene knocked out and has been contacted with the a compound that promotes neurogenesis; and
c) determining the effect of the compound that promotes neurogenesis on the expression of the reporter protein in the neurons, such that if there is an increase in the number of neurons expressing a reporter protein in the zebrafish that does not have a neuron-specific gene knocked out compared with the number of neurons expressing a reporter protein in a transgenic zebrafish with a neuron-specific gene knocked out the neuron-specific gene is a target for the compound that promotes neurogenesis.

27-29. (canceled)

30. A method of identifying a neuron-specific gene as a target for a compound that regenerates neurons comprising:
a) contacting a neuronally damaged transgenic zebrafish expressing a reporter protein in neurons and has a neuron-specific gene knocked out with a compound that regenerates neurons;
b) comparing the expression of the reporter protein in the neurons of the transgenic zebrafish with a neuron-specific gene knocked out with the expression of the reporter protein in the neurons of a neuronally damaged transgenic zebrafish that does not have a neuron-specific gene knocked out and has been contacted with a compound that regenerates neurons; and determining the effect of the compound that regenerates neurons on the expression of the reporter protein in the neurons, such that if expression of the reporter protein in the zebrafish that does not have a neuron-specific gene knocked out is greater than the expression of the reporter protein in a transgenic zebrafish with a neuron-specific gene knocked out the neuron-specific gene is a target for the compound that regenerates neurons.

31-32. (canceled)

34. A method of identifying a neuroprotective compound that effects neuronal protection via a neuron-specific gene comprising:
a) contacting a transgenic zebrafish that expresses a reporter protein in neurons and has a neuron-specific gene knocked out with a neurotoxin and test compound;
b) comparing the expression of the reporter protein in the neurons of the transgenic zebrafish with a neuron-specific gene knocked out with the expression of the reporter protein in the neurons of a neuronally damaged transgenic zebrafish that does not have a neuron-specific gene knocked out and has been contacted with the test compound; and
c) determining the effect of the test compound on expression of the reporter protein in neurons, such that if expression of the reporter protein in the neurons of the zebrafish contacted with the neurotoxin and the test compound is greater than the expression of the reporter protein in the transgenic zebrafish that expresses a reporter protein in neurons and has a neuron-specific gene knocked out, the compound is a neuroprotective compound that effects neuroprotection via the neuron-specific gene that has been knocked out.

35-37. (canceled)

38. A method of identifying a compound that regenerates neurons via a neuron specific gene comprising:
a) contacting a neuronally damaged transgenic zebrafish that expresses a reporter protein in neurons and has a neuron-specific gene knocked out with a test compound;
b) comparing the expression of the reporter protein in the neurons of the transgenic zebrafish with a neuron-specific gene knocked out with the expression of the reporter protein in the neurons of a neuronally damaged transgenic zebrafish that does not have a neuron-specific gene knocked out and has been contacted with the test compound; and
c) determining the effect of the test compound on expression of the reporter protein in neurons, such that if expression of the reporter protein in the neurons of the zebrafish contacted with the test compound is greater than the expression of the reporter protein in the transgenic zebrafish that expresses a reporter protein in neurons and has a neuron-specific gene knocked out, the compound is a compound that regenerates neurons via the neuron-specific gene that has been knocked out.

39-41. (canceled)

42. A method of identifying a compound that promotes neurogenesis via a neuron-specific gene comprising:
a) contacting a neuronally damaged transgenic zebrafish that expresses a reporter protein in neurons and has a neuron-specific gene knocked out with a test compound;
b) comparing the expression of the reporter protein in the neurons of the transgenic zebrafish with a neuron-specific gene knocked out with the expression of the reporter protein in the neurons of a neuronally damaged transgenic zebrafish that does not have a neuron-specific gene knocked out and has been contacted with the test compound; and;
c) determining the effect of the test compound on expression of the reporter protein in neurons, such that if there is an increase in the number of neurons expressing the reporter protein in the zebrafish contacted with the test compound compared with the number of neurons expressing a reporter protein in the transgenic zebrafish that expresses a reporter protein in neurons and has a neuron-specific gene knocked out, the compound promotes neurogenesis via the neuron-specific gene that has been knocked out.

43-49. (canceled)

50. A method of obtaining a gene associated with neuroprotection comprising: mutagenizing a transgenic zebrafish that expresses a reporter protein in neurons and exhibits neuroprotection in the presence of a neurotoxin and a neuroprotectant
a) administering a neurotoxin and the neuroprotectant to the mutagenized transgenic zebrafish of a);
b) comparing the expression of the reporter protein in the neurons of the transgenic zebrafish of b) with the expression of the reporter protein in the neurons of an unmutagenized transgenic zebrafish that expresses a reporter protein in neurons and exhibits neuroprotection in the presence of a neurotoxin and the neuroprotectant compound;
c) determining the effect of the test compound on expression of the reporter protein in neurons, such that if there is change in neuroprotection in the mutagenized zebrafish as compared to the unmutagenized zebrafish, the mutagenized zebrafish contains a mutation in a gene associated with neuroprotection;
d) mapping the mutant gene; and
e) cloning the gene associated with neuroprotection.

51. A method of obtaining a gene associated with regeneration comprising:
a) mutagenizing a transgenic zebrafish that expresses a reporter protein in neurons and that when neuronally damaged exhibits regeneration in the presence of a compound that promotes regeneration;
b) neuronally damaging the mutagenized transgenic zebrafish of a);
c) administering a compound that promotes regeneration;
d) comparing the expression of the reporter protein in the neurons of the transgenic zebrafish of b) with the expression of the reporter protein in the neurons of an unmutagenized transgenic zebrafish that expresses a reporter protein in neurons and that when neuronally damaged exhibits regeneration in the presence of a compound that promotes regeneration;
e) determining the effect of the test compound on expression of the reporter protein in neurons, such that if there is change in regeneration in the mutagenized zebrafish as compared to the unmutagenized zebrafish, the mutagenized zebrafish contains a mutation in a gene associated with regeneration;
f) mapping the mutant gene; and
g) cloning the gene associated with regeneration.

52. A method of obtaining a gene associated with neurogenesis comprising:
a) mutagenizing a transgenic zebrafish that expresses a reporter protein in neurons and that when neuronally damaged exhibits neurogenesis in the presence of a compound that promotes neurogenesis;
b) neuronally damaging the mutagenized transgenic zebrafish of a);
c) administering a compound that promotes neurogenesis;
d) comparing the expression of the reporter protein in the neurons of the transgenic zebrafish of c) with the expression of the reporter protein in the neurons of an unmutagenized transgenic zebrafish that expresses a reporter protein in neurons and that when neuronally damaged exhibits neurogenesis in the presence of a compound that promotes neurogenesis;
e) determining the effect of the test compound on expression of the reporter protein in neurons, such that if there is change in the number of neurons expressing a reporter protein in the mutagenized zebrafish as compared to the unmutagenized zebrafish, the mutagenized zebrafish contains a mutation in a gene associated with neurogenesis;
f) mapping the mutant gene; and
g) cloning the gene associated with neurogenesis.

53. A method of identifying a neuron-specific gene comprising:
a) Constructing a neuron-specific zebrafish library;
b) Identifying a neuron-specific zebrafish gene.

54-55. (canceled)


SEQUENCE LISTING (1 sequence)

SEQ ID NO: 1 - DNA, 13 bp, Artificial Sequence
  Restriction Enzyme
ggccnnnnnggcc